(19) United States
(12) Patent Application Publication
(10) Pub. No.: US 2011/0033661 A1
(43) Pub. Date: Feb. 10, 2011

(54) CONTROLLABLE NANOSTRUCTURING ON MICRO-STRUCTURED SURFACES

(75) Inventor: Takahiro Oawa, Los Angeles, CA (US)

Correspondence Address: SQUIRE, SANDERS & DEMPSEY L.L.P., 275 BATTERY STREET, SUITE 2600, SAN FRANCISCO, CA, 94111-3356, US

(73) Assignee: THE REGENTS OF THE UNIVERSITY OF CALIFORNIA, Oakland, CA (US)

(21) Appl. No.: 11/909,156

(22) PCT Filed: Mar. 21, 2006
(86) PCT No.: PCT/US06/10281

§ 371 Date: Jul. 15, 2009

Related U.S. Application Data
(60) Provisional application No. 60/664,243, filed on Mar. 21, 2005.

(51) Int. Cl. B32B 3/00; C23C 16/44; B24C 1/00; C23F 1/00; B82Y 40/00; B82Y 5/00; B82Y 30/00

(52) U.S. Cl. 428/141; 427/248.1; 451/38; 216/58; 977/773; 977/891; 977/888; 977/904; 977/932

(57) ABSTRACT

Provided herein is a medical implant having a nanostructure on top of a microstructure and the methods of making and using the same.

### BACKGROUND OF THE INVENTION

1. Field of the Invention

This invention generally relates to a process for creating nano-sphere structures on micro-structured surfaces.

2. Description of the Background

Nanostructuring and/or nano-coating technology have proven to create unique physical (He, G., et al., Nat Mater 2, 33-7 (2003)), chemical, mechanical (He, G. et al. Biomaterials 24, 5115-20 (2003); Wang, Y., et al., Nature 419, 912-5 (2002)) and biological properties (Webster, T. J., et al., Biomaterials 20, 1221-7 (1999)) of various materials, which explores next generation of the existing micron-scale technologies for extensive potential applications in the fields of engineering, information technology, environmental sciences and medicine. There are two common strategies for creating nano-surface structures: 1) the so-called top-down approach and 2) the bottom-up approach. Since the top-down approach, represented by the submicron level laser lithography, is to create nanostructures from the macro- and micro-basically by subtractive modification of original surfaces, the size of the processed structure is dependent on the resolution and wave length of the beam source. Moreover, this time-consuming approach is not suitable for large-scale processing and mass production. In contrast, the bottom-up approach creates nanostructures from pico- and sub-nano-levels, as represented by atomic assembly using a nano-level-resolution microscopy and metal solidification. The bottom-up type of nanostructuring is expected to overcome the limitation of the top-down methods by improving the processing scale, speed and cost. However, currently available technologies do not overcome the rapid, controllable and low-cost nanostructuring of large surfaces or interfaces, e.g., an area equal to or larger than 1 mm² scale. Another issue is that the current technologies have difficulties in creating a co-existence of microstructure and nanostructure, which gives additional properties of the new surface maintaining the existing micro-structure. For instance, in bioengineering fields, it would be beneficial to increase the surface area and roughness of biomaterials without altering the existing micro-scale configuration, which may help enhance protein-biomaterial interaction without sacrificing favorable cell-biomaterial interaction. An example of such cell-biomaterial interaction is bone-titanium integration, an essential biological phenomenon for orthopedic and dental implant treatments. The bone cell-affinitive implant surfaces have been established at a micron level, and a current challenge is to add molecule-affinitive structure without changing the established surface.

There is a great need for faster and stronger fixation and reconstruction of bone, joints and teeth by metallic and non-metallic implants (such as zirconia implants). The embodiments described below address the above identified issues and needs.

### SUMMARY OF THE INVENTION

Provided herein is a substrate surface structure having a surface that has a nanostructure and a microstructure. The substrate surface structure is generated by a controlled nanostructuring process that allows the creation of nanostructure on the top of the existing microstructure on the surface of the substrate. The nanostructuring process described herein can be, e.g., a vapor deposition process such as electron-beam physical vapor deposition (EB-PVD). Other useful deposition processes include, but are not limited to, sputter coating, electric current, heat-, laser- and ultrasound-vapor deposition, plasma spray, ion plating and chemical vapor deposition based on e.g., photo-, heat-, gas-, and chemical-driven reaction.

The nanostructuring process can be used to create a nanostructured substrate surface structure on any substrate. The substrate can be any article, e.g., a medical or a biomedical article formed of a metallic material, a non-metallic material, or a polymeric material. For example, the article can be a medical implant or a semiconductor article. One such medical implant is a titanium implant.

### BRIEF DESCRIPTION OF DRAWINGS

FIGS. 1a-1c show the creation of nano-sphere structure of titanium on pre-micro-roughened titanium.

FIGS. 2a-2d show control of nano-sphere structure by altering deposition time.

FIG. 3 shows scanning electron micrographs showing Ti nano-spheres created on non-metal surfaces.

FIG. 4 shows ceramic and semiconductor nano structuring.

FIG. 5 shows scanning electron micrographs after Ti electron-beam physical vapor deposition (EB-PVD) on variously modified alloys, nickel and chromium surfaces.

FIG. 6 shows nanostructuring between heterogeneous metals.

FIG. 7 shows nanostructuring of Ti surface using a different deposition technique.

FIG. 8 shows a formation of nanospheres on the zirconium dioxide surface.

FIG. 9 shows the nanostructure-enhanced bone-titanium integration evaluated by biomechanical push-in test.

### DETAILED DESCRIPTION

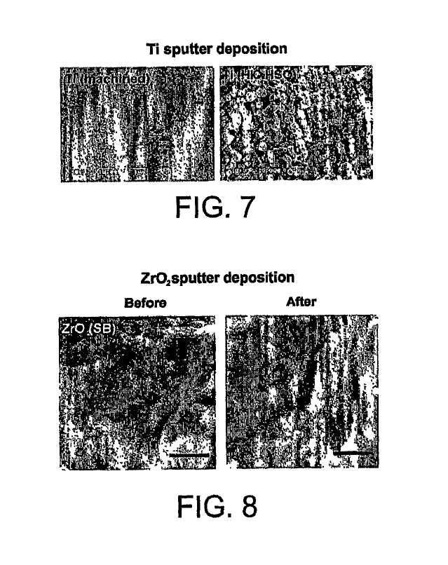

Provided herein is a substrate surface structure having a surface that has a nanostructure and a microstructure. The substrate surface structure is generated by a process that allows the generation of a nanostructure on the top of an existing microstructure on the surface of a substrate. Generally, the process includes: (a) forming a microstructure on a substrate, and (b) forming a nanostructure on top of the microstructure by a controlled nanostructuring process. The step of forming a microstructure can be a physical process, a chemical process, or a combination thereof, which are further described below. The step of forming a nanostructure can be, e.g., a vapor deposition process such as electron-beam physical vapor deposition (EB-PVD). Other useful deposition processes include, but are not limited to, sputter coating (see FIG. 7; see also Ding et al., Biomaterials 24, 4233-8 (2003)), electric current, heat-, laser- and ultrasound-vapor deposition (Wagner, J Oral Implantol 18, 231-5, (1992)), plasma spray (Xue et al., Biomaterials 26, 3029-37 (2005)), ion plating (McCrory et al., J Dent 19, 171-5 (1991)) and chemical vapor deposition based on e.g., photo-, heat-, gas-, and chemical-driven reaction (Lamperti et al., J Am Soc Mass Spectrum 16, 123-31 (2005)).

Nano-level roughness provides approaches for more intimate interlocking between hetero-metals and between metal and other materials, leading to many applications. For example, an increased surface area by nanostructuring can boost ability of electrodes and batteries. Nanostructure, including nano-pore, nano-size particles, nano-scale gap and precisely controlled interface, may act as a thermal barrier to reduce device's energy demand and to add nano-scale functionality, such as DNA/nanostructure complex. Since organic and inorganic components of biological tissue stand in nanoscale, nanostructured metal would have more affinitive interaction with cells, not only because the metal mimics the fundamental scale of constituent components of surrounding tissue (concept of molecular mimetics) (Sarikaya, M., et al., Nat Mater 2, 577-85 (2003)) but also nano-level molecular interlocking of the metal surface and matrix molecules.

The process described herein can be used to create a nanostructured substrate surface structure on any substrate. The substrate can be any article, e.g., a medical or a biomedical article formed of a metallic material, a non-metallic material, or a polymeric material. For example, the article can be a medical implant or a semiconductor article. One such medical implant is a titanium implant.

In some embodiments, the nanostructure contains nanoparticles or nanospheres that do not form a continuous phase, for example, the naonospheres or nanoparticles can form a non-continuous phase.

### Controlled Nanostructuring

The controlled nanostructuring process described herein generally includes the steps of (1) causing the formation of a vapor of a nanostructuring material, (2) depositing the vapor on a substrate having a microstructure surface, and (3) forming a nanostructure of the nanostructuring material on the substrate on the microstructure surface.

There are many established method of causing a nanostructuring material to vaporize. The three basic vapor deposition techniques are: evaporation, sputtering, and chemical vapor deposition. The nanostructuring material can be vaporized with or without vacuum. The source of vapor energy can be thermal control, ion and electron beams, electrical current, ultrasound, laser, gas, photo and chemicals.

The step of depositing can be direct deposit and other deposition processes with thermal, electrical and pressure controls. The surface energy of substrates can also be controlled.

Some exemplary methods of deposition include, but are not limited to, sputter coating, thermal vapor coating, plasma spraying, and electron-beam physical vapor deposition (EB-PVD) technology, chemical vapor deposition technology, ion plating and combinations thereof.

The nanostructure on the substrate can be in any physical appearance. In one embodiment, the nanostructure can be a plurality of nano-spheres or nanoparticles. The nanostructure generally has a size in the range from about 1 nm to over 1000 nm, e.g., about 5 nm, about 10 nm, about 20 nm, about 50 nm, about 80 nm, about 90 nm, about 95 nm, about 100 nm, about 200 nm, about 500 nm, about 800 nm, about 900 nm, about 1000 nm or about 1500 nm. The size of the nanostructure can be controlled by e.g., controlling the density of the vapor of the nanostructuring material, the rate of deposition, and deposition time. The density of the vapor positively relates to the degree of vacuum and strength of energy sources. The rate of deposition can be controlled by, e.g., the strength of energy sources.

The substrate can be subjected to surface treatment to acquire a microstructure prior to the application of the process described herein. The surface treatment can be a physical process such as machining or sand-blasting, or a chemical process such etching with a chemical agent such as an acid or base, thermal oxidation or anodic oxidization, or combinations thereof.

The nanostructuring process described herein can be used to generate substrates in many different fields. For example, this process have applications in the development of electronically, optically, chemically and mechanically modified/optimized materials and interfaces, molecular recognition technology, and more biocompatible tissue engineering and implantable materials.

In the nanostructuring process, the nanostructuring material can be the same or different from the material forming the substrate. For example, titanium can be used as a nanostructuring material on a substrate formed of titanium or a non-titanium material. Selection of a nanostructuring material for a particular substrate depends on and can be readily determined by the application or use of a substrate.

### Nanostructuring Materials

The nanostructuring material forming the nanostructures on a substrate can be any nanostructuring material. For example, the nanostructuring material can be a metal such as a noble metal e.g., gold, platinum, or an alloy thereof, etc, or a biocompatible metal or alloy e.g., titanium, zirconium or an alloy including titanium alloy and chromium-cobalt alloy, or oxidized metal including titanium dioxide or zirconium dioxide. The nanostructuring material can also be non-precious metals e.g., nickel, chromium, cobalt, aluminum, copper, zinc, ferrous, cadmium, lithium, or an alloy thereof, or an oxided metal including aluminum oxide. In some other embodiments, the nanostructuring material can be a semiconductor material silicon, silicon dioxide, GaAs, or other semiconductor materials, or ceramic material, including aluminum oxide, magnesium oxide, silicon dioxide, silicon carbonate, or plastic materials including polystyrene. In some other embodiments, the nanostructuring material can be an organic or polymeric material for forming biocompatible nanostructures on top of a substrate, e.g., PLA (poly lactic acid), PLGA (poly lactic-co-glycolic acid), poly methyl methacrylate (PMMA), silicone, silicone acrylate, polytetrafluoroethylene (PTFE), Teflon, stainless steel, poly urethane, cellulose, and apatite and other calcium phosphate. In some embodiments, the nanostructuring material can be a bioglass.

In some embodiments, the nanostructuring material can specifically exclude any of the above described materials. For example, the nanostructuring material can exclude a ceramic or ceramics such as apatite or any calcium phosphate compounds or a metal oxide such as aluminum oxide. As used herein, the term ceramic does not include a metal oxide such as zirconium oxide.

### Substrates

The substrates described herein can be any articles. In some embodiments, the substrate can be an article formed of a metallic material which can be elemental metal or a metal alloy or a non-metallic material such as semiconductor, ceramic material or polymeric material or combinations thereof. The substrate can have a microstructure surface.

The substrate formed of a metallic material can be, for example, an implant formed of a biocompatible metallic material such as materials comprising titanium, zirconium or an alloy including titanium alloy and chromium-cobalt alloy, or oxidized metal including titanium dioxide or zirconium dioxide.

The substrate described herein can also be non-precious metals e.g., nickel, chromium, aluminum, copper, zinc, ferrous, cadmium, lithium, or an alloy thereof, or oxide metal including aluminum oxide. In some other embodiments, the substrate can be a semiconductor material such as silicon, silicon dioxide, GaAs, or other semiconductor materials, an oxide material such as zirconium dioxide, aluminum oxide, magnesium oxide, silicon dioxide, silicon carbonate, or a plastic material including polystyrene. In some other embodiments, the substrate allowing the nanostructures can be an organic, inorganic or polymeric material for forming biocompatible nanostructures on top of the substrate, e.g., PLA (poly lactic acid), PLGA (poly lactic co-glycolic acid), collagen, poly methyl methacrylate (PMMA), silicone, silicone acrylate, polytetrafluoroethylene (PTFE), Teflon, stainless steel, poly urethane, cellulose, and apatite and other calcium phosphate.

The medical implant described herein can be porous or non-porous implants. Porous implants generally have better tissue integration while non-porous implants have better mechanical strength.

The substrate formed of a non-metallic material can be, polymeric implants, biomedical graft material, tissue engineering scaffolds, etc., formed of a biocompatible polymeric material such as PLA (poly lactic acid), PLGA (poly lactic co-glycolic acid), poly methyl methacrylate (PMMA), silicone, silicone acrylate, polytetrafluoroethylene (PTFE), Teflon, stainless steel, poly urethane, cellulose, and apatite and other calcium phosphate.

### Surface Treatment

Prior to the nano-structuring described above, the substrate is subject to surface treatment to generate a microstructure on the surface of the substrate. Such surface treatment can be any suitable chemical or physical treatment or treatments capable of creating a microstructure on the substrate surface. Suitable physical treatments include, e.g., machining, sand-blasting, sand-paper grinding or heating. Suitable electro-chemical treatments include anodic oxidation, photo-chemical-etching and discharge processing. Suitable chemical treatments include, e.g., etching by a chemical agent such as an acid or a base or anodic oxidization. Representative useable acids include any inorganic acid such as HCl, HF, HNO₃, H₂SO₄, H₂SiF₆, CH₃COOH, H₃PO₄, C₂H₄O₂ or a combination thereof. Representative useable base include, e.g., NaOH, KOH, Na₂CO₃, K₂CO₃, NH₄OH, or a combination thereof.

### Method of use

The nano-structured substrates described herein can have many applications. In one embodiment, the nano-structured substrate is a nano-structured metallic and ceramics article which has improved chemical, physical, mechanical, electronic, thermal and biological properties. In another embodiment, the nano-structured substrate is a thin silicon dioxide coating. Thin silicon dioxide coating can improve the properties of gas barrier, electronic insulation, gas sensors. In still another embodiment, the nano-structured substrate is a Ti catalyst, of which photocatalytic activity of Ti is made more effective and efficient by its increased surface area by the nano-spheres thereon. In still another embodiment, the nano-structured titanium can be an osseous implant material for improved bone, and/or joint and tooth anchorage and reconstruction.

### EXAMPLES

The embodiments of the present invention will be illustrated by the following set forth examples. All parameters and data are not to be construed to unduly limit the scope of the embodiments of the invention.

### Example 1

### Formations of Nano-Spheres on Various Substrates

General Methods

Substrate Preparation

Surfaces of commercially pure titanium, nickel and chromium, titanium alloy (Ti 85.5%, Al 6.0%, Nb 7%), chromium cobalt alloy, and zirconium dioxide were prepared by either machining, sand-blasting (25 μm or 50 μm AlO₂ particles for 1 min at a pressure of 3 kg/m), various acid-etching using 66.3% H₂SO₄ at 115° C. for 1 min, 10.6% HCl at 70° C. for 5 min, 3% HF at 20° C. for 3 min, Chromium etchant (5-10% HNO₃, 1-5% H₂SO₄, 5-10% ceric sulfate) at 40° C. for 15 min, nickel etchant (70% HNO₃) at 25° C. for 20 min, or a combination of these. Additionally, non-metal substrates, including the polystyrene cell culture dishes, microscopic slide glasses, poly-lactic acid (PLA) and collagen membrane (Ossix, Implant Innovations, Inc, Palm Beach, Fla.) and silicon wafer.

Metallic Deposition

Surfaces of the prepared substrates were deposited with either titanium, nickel or chromium using e-beam physical vapor deposition (EB-PVD) technology (SLONE e-beam evaporator, SLONE Technology Co. Santa Barbara, Calif.). The deposition rate was 3 Å/s for Ti, Ni, Cr, SiO₂, and 2 Å/s for Si to the calculated final thickness of deposition of 100 nm, 250 nm, 500 nm, or 1000 nm. Titanium deposition and zirconium dioxide deposition were also attempted using a sputtering technology (Sputter Deposition System CVC 601) with a deposition rate of 1.3 Å/s.

Surface Characterization

Surface morphology was examined by scanning electron microscopy (SEM) (JSM-5900LV, Joel Ltd, Tokyo, Japan) and atomic force microscopy (AFM) (SPM-9500J3, Shimadzu, Tokyo, Japan). The contact mode scanning was performed in the area of 5 μm×5 μm, and the images were constructed with a custom vertical scale. The AFM data were analyzed using packaged software for topographical parameters of average roughness (Ra), root mean square roughness (Rrms), maximum peak-to-valley length (Rp-v) and inter-irregularities space (Sm).

Animal Surgery

Five 8-week-old male Sprague-Dawley rats were anesthetized with 1-2% isoflurane inhalation. After their legs were shaved and scrubbed with 10% providone-iodine solution, the distal aspects of the femurs were carefully exposed via skin incision and muscle dissection. The flat surfaces of the distal femurs were selected for implant placement. The implant site was prepared 9 mm from the distal edge of the femur by drilling with a 0.8 mm round burr followed by reamers #ISO 090 and 100. Profuse irrigation with sterile isotonic saline solution was used for cooling and cleaning. One untreated cylindrical acid-etched implant and one nano-structured acid-etched implant were placed into the right and left femurs, respectively. The University of California at Los Angeles (UCLA) Chancellor's Animal Research Committee approved this protocol and all experimentation was performed in accordance with the United States Department of Agriculture (USDA) guidelines of animal research.

Implant Stability Test

This method to assess biomechanical strength of bone-implant integration is described elsewhere (Ogawa et al., 2000). Briefly, femurs containing a cylindrical implant were harvested and embedded immediately in auto-polymerizing resin with the top surface of the implant level. The testing machine (Instron 5544 electro-mechanical testing system, Instron, Canton, Mass.) equipped with a 2000 N load cell and a pushing rod (diameter=0.8 mm) was used to load the implant vertically downward at a crosshead speed of 1 mm/min. The push-in value was determined by measuring the peak of load-displacement curve.

A. Nano-Spherical Structures of Titanium

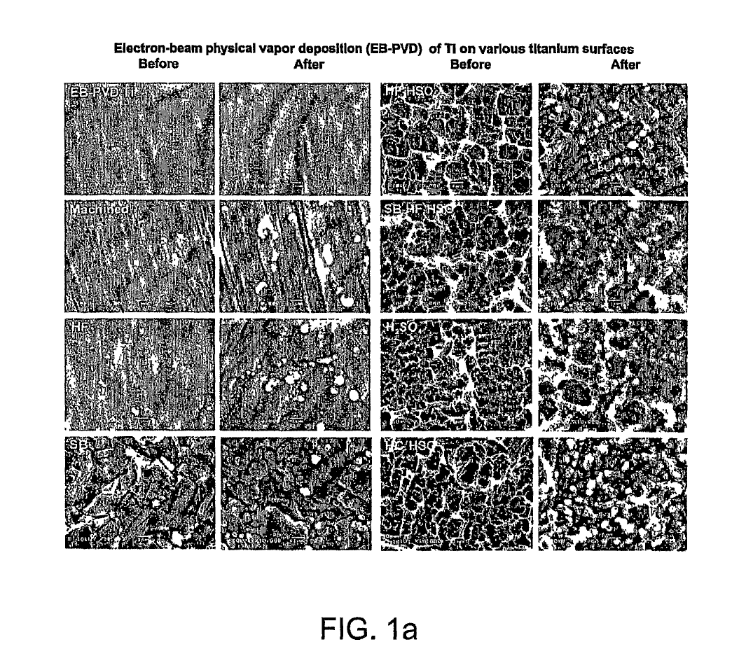

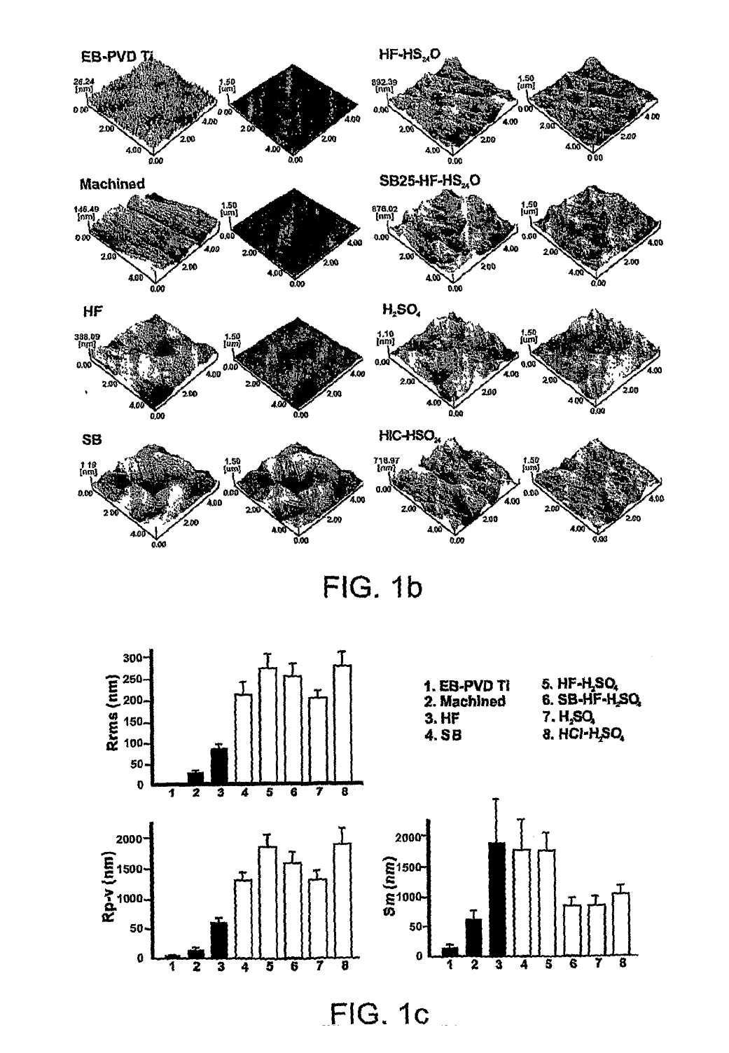

Nano-spherical structures were created by electron-beam physical vapor deposition (EB-PVD) on variously prepared Ti surfaces. Titanium is the most biocompatible metal used extensively as orthopedic and dental implants, and widely noticed for new applications owing to its photo-catalytic activity. Scanning electron micrographs revealed that uniform nanostructuring only occurred on roughened surfaces by either sand-blasting, acid-etching using various chemicals, or a combination of these (FIG. 1a). FIG. 1a shows scanning electron micrographs before and after electron-beam physical vapor deposition (EB-PVD) of titanium on various titanium surfaces showing the emergence of Ti nanostructure. The deposition time was 16 minutes 40 seconds for all. Titanium was deposited on either EB-PVD titanium coated polystyrene, machined surface, hydrofluoric acid etched surface (HF), sand-blasted with 25 μm aluminum oxide (SB25), hydrofluoric acid and sulfuric acid dual etched surface with (SB25-HF—H₂SO₄) or without (HF—H₂SO₄) pre-sand-blasting, sulfuric acid etched surface (H₂SO₄), and hydrochloric acid and sulfuric acid dual etched surface (HCl—H₂SO₄). The gray highlighted images indicate no or little nano-sphere structure created, while the blue highlighted images indicate dense, uniform and consistent ones.

The morphology and density of nano-spheres differed among the different substrate modification. The nanostructures were more even and uniform on the acid-etched substrates than on the sand-blasted substrates, in accordance with the evenness of roughness on the substrates. The substrates morphology before Ti EB-PVD was evaluated by the atomic force microscopy (AFM) (FIG. 1b). FIG. 1b shows atomic force micrographs of the various Ti substrates tested showing various degree of micro-roughness before titanium electron-beam physical vapor deposition (EB-PVD). The images are presented in two different vertical scales; maximum peak for each substrate (left lane) and 1.5 μm (right lane). The AFM images in a custom vertical scale exhibited various nature of roughness for every substrate tested, while the images in a fixed vertical scale of 1.5 μm showed the recognizable roughness only for the sand-blasted (SB), HF-H₂SO₄, SB—HF—H₂SO₄, H₂SO₄, or HCl—H₂SO₄ treated surfaces, all of which created the nano-sphere structure afterward. Quantitative measurement of the surface roughness of the substrates indicated that emergence of the nanosphere structures were associated with the substrate surface topography that was >200 nm in the root mean square roughness (Rrms) and >1000 nm in the maximum peak-to-valley length (Rp-v) (FIG. 1c). FIG. 1c shows roughness analysis for the substrates before the Ti deposition. Data is shown as a mean and standard deviation (n=3). There seemed to be no requirements for an inter-irregularities space (Sm): Sm around 1000 nm seemed to help develop the dense nanospheres compared to Sm greater than 1500 nm. These indicate that the existing micro-level surface roughness with appropriate dimensions is a prerequisite for the nano-sphere structuring described herein.

B. Controlled Formation of Nano-Spheres

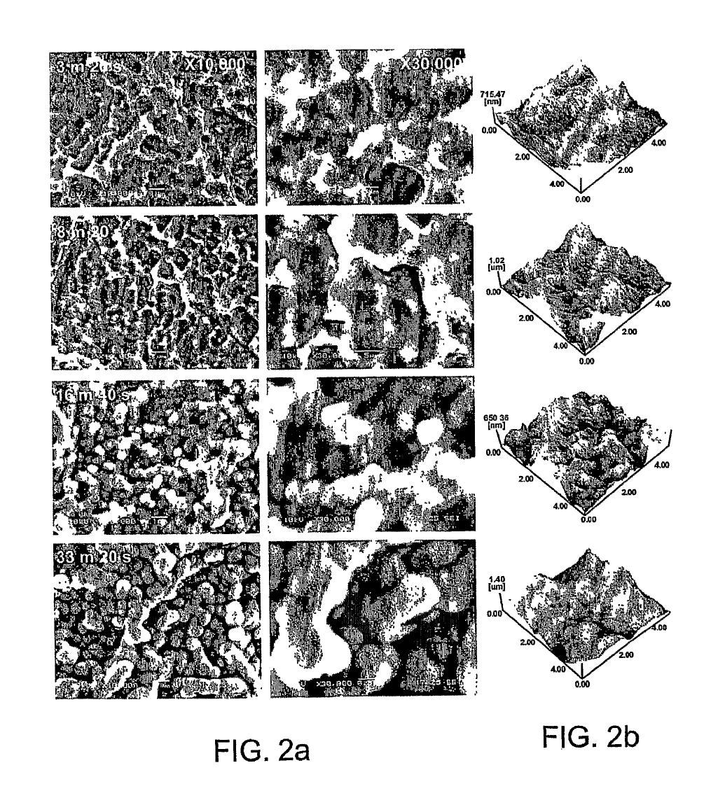

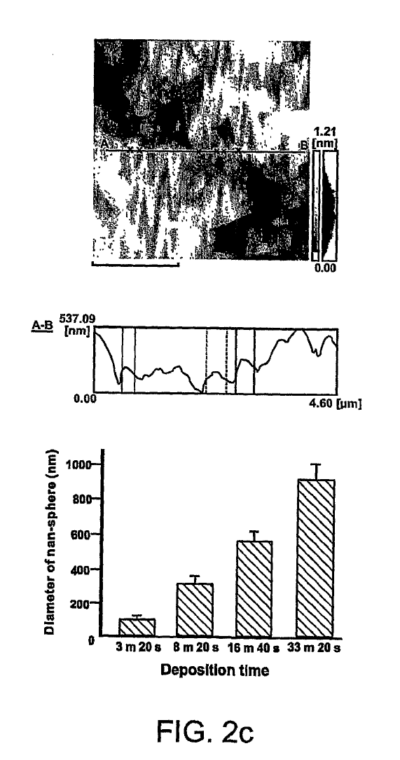

Nano-spheres were formed with controlled sizes. FIGS. 2a-2d shows evolution of the nano-sphere with an increase of deposition time. Ti EB-PVD was performed on the HCl—H₂SO₄ acid etched Ti surface with different deposition time. When the deposition time was 3 minutes 20 seconds with a deposition rate of 5 Å/s, development of nanospheres having a size under 100 nm, of which averaged diameters are 84 nm, was recognizable. Increased deposition time grew the nanospheres larger, even greater than 1000 nm in diameter with the average diameter of 925 nm (FIG. 2a). FIG. 2a shows the scanning electron micrographs after Ti electron-beam physical vapor deposition (EB-PVD) for various deposition time, showing the size of nano-spherical structures correlated to the deposition time. The deposition rate was fixed at the 0.3 nm/s. The averaged size of the developed nanospheres, ranging from 84 nm to 925 nm, was in linear correlation with the deposition time we tested (FIGS. 2b and 2c). FIG. 2b shows the atomic force micrographs of the deposited Ti surface. FIG. 2c shows the measurement of the diameter of the nano-spheres (data is shown as a mean and standard deviation (n=9)). The co-existence of the substrate microstructure, represented morphologically by its peaks and valleys, and the nano-spheres added along the flank of the roughness or in the valley was clearly seen when the deposition time was 8 minutes 20 seconds or less (FIG. 2d).

C. Nano-Spheres of a Metallic Material on Non-Metallic Substrates

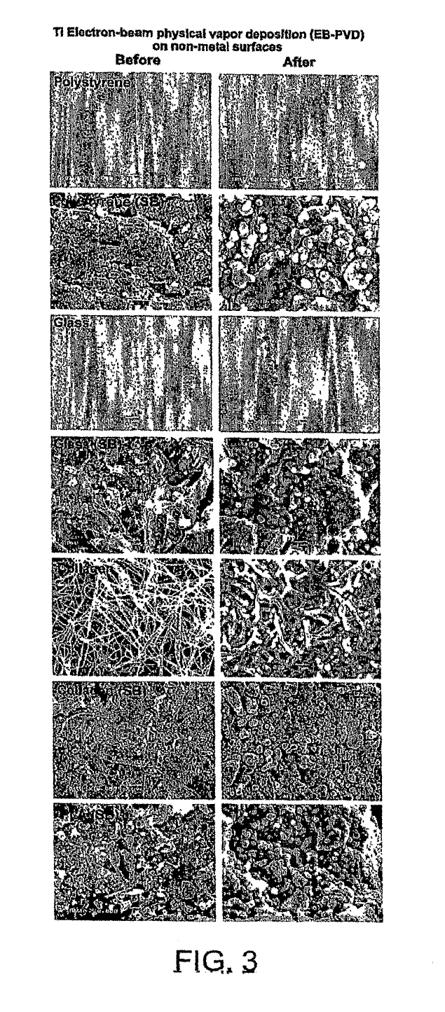

To determine a possibility of metal nanostructuring on non-metal surfaces, the Ti EB-PVD was applied onto non-organic materials of polystyrene and glass, and bioabsorbable tissue engineering materials of collagen membrane and poly-lactic acid (PLA) (FIG. 3). Ti nanostructures similar to those on the metal surfaces were constructed on the all of the nonmetals tested, when they were pre-roughened by sand-blasting. In the test shown by FIG. 3, Ti was deposited onto the original surface or sand-blasted surface of polystyrene, glass, collagen membrane and poly-lactic acid (PLA) using electron-beam physical vapor deposition (EB-PVD).

D. Nano-Spheres Formed of Non-Metallic Materials

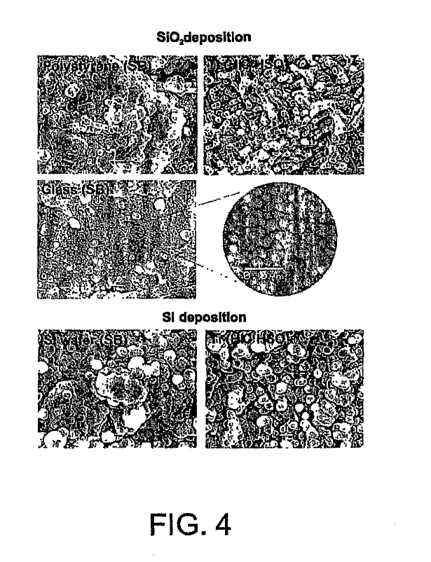

Nano-spherical structures of ceramic and semiconductor materials can be generated according to the method described herein (FIG. 4). Both SiO₂ and Si EB-PVD generated their nano-spheres on the metallic and non-metallic substrates, including Si wafers, as long as the substrates were micro-roughened. In the test shown by FIG. 4, Scanning electron micrographs showing SiO₂ and Si nano-spheres created on metal and non-metal surfaces. SiO₂ or Si was deposited using electron-beam physical vapor deposition (EB-PVD) onto the original surface or sand-blasted surface of polystyrene and glass, Si wafer and machined or acid etched (HCl—H₂SO₄) titanium surfaces.

E. Nano-Spheres Generated on Different Metal Surfaces

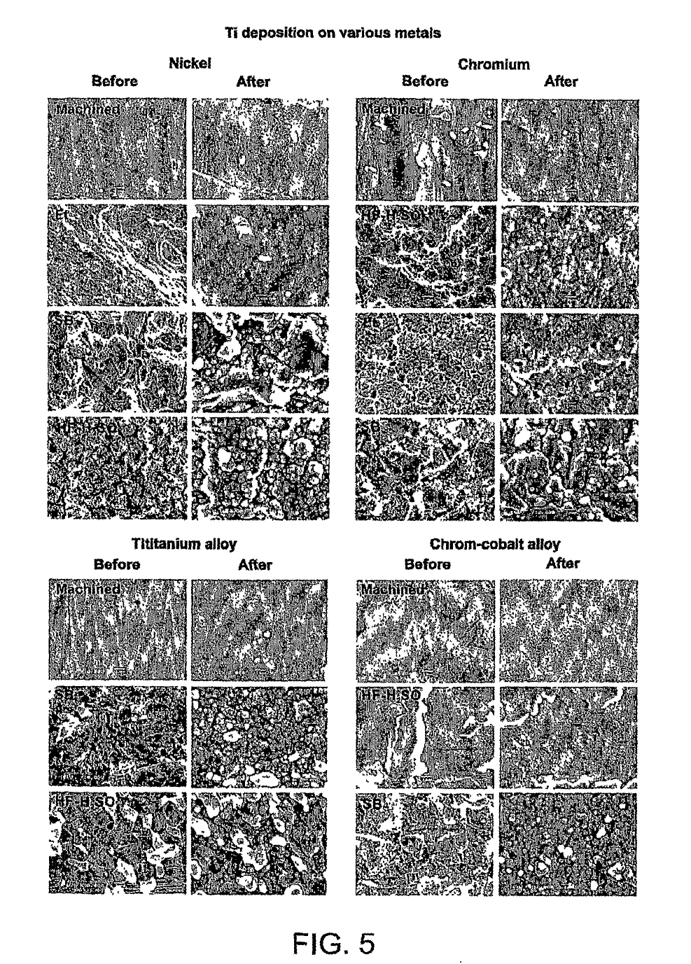

Nano-spheres of titanium or a metal than titanium and nano-spheres of a metallic material on the substrate of a different metal or metals were generated. FIG. 5 shows successful creation of Ti nanostructures on the sand-blasted and acid-etched Ni and Cr. Ti nanospheres on Ti alloy or Co—Cr alloy, both are well-known biocompatible alloys, were created when the alloys' surfaces were micro-roughened by sand-blasting or acid-etching. The surfaces were prepared by machining (Machined), sand-blasting with 25 μm aluminum oxide (SB25), hydrofluoric acid and sulfuric acid dual etching (HF—H₂SO₄), or commercially available etchant (Et). The gray highlighted images indicate no or little nano-sphere structure created, while the blue highlighted images indicate dense, uniform and consistent ones.

F. Nano-Spheres Formed of Chromium or Nickel

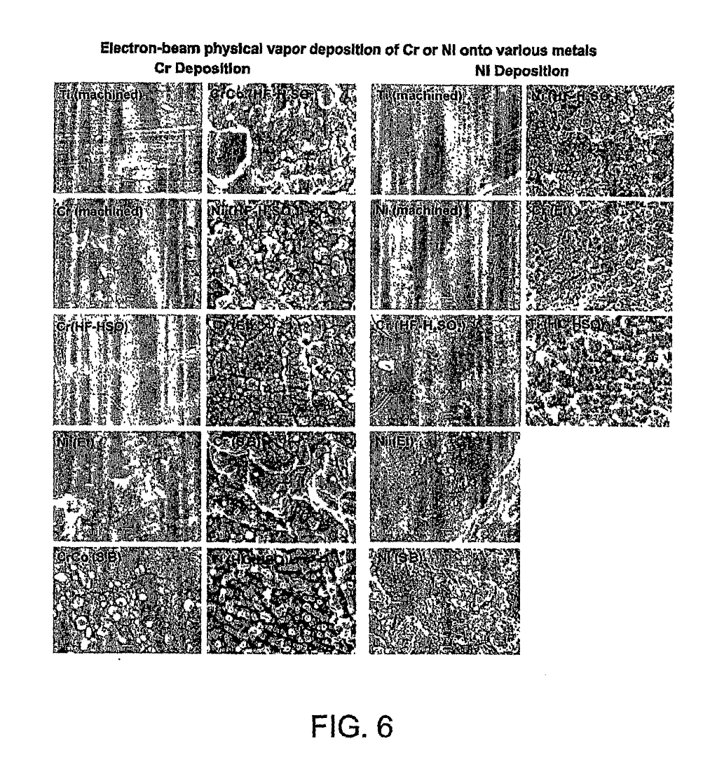

Nano-spheres formed of chromium or nickel can be generated on roughened surfaces of different metallic substrates. FIG. 6 shows nano-spheres of Cr and Ni on microstructured (micro-roughened) surfaces of various metals, indicating that the nanostructuring on microstructured surfaces can be formed between heterogeneous metals, showing that there is no restriction on the type of materials for nanostructuring (forming nano-spheres) nor on the substrates being nano-structured. The surfaces were prepared by machining (Machined), sand-blasting with 25 μm aluminum oxide (SB25), hydrofluoric acid and sulfuric acid dual etching (HF—H₂SO₄), or commercially available etchant (Et). The gray highlighted images indicate no or little nano-sphere structure created, while the blue highlighted images indicate dense, uniform and consistent ones.

G. Nano-Spheres Formed Using a Different Deposition Technique

A sputtering technology was also employed to deposition titanium onto the acid-etched titanium surface. FIG. 7 shows the generated nano-spherical structure on the acid-etched surface but not on the machined surface, indicating the successful nano-sphere formation of material surfaces and interfaces using various vapor deposition techniques. In FIG. 7, scanning electron micrographs are presented after Ti sputter coating on the machined Ti or acid-etched Ti (HCl—H₂SO₄). The gray highlighting is for unsuccessful nano-sphere structuring, while the blue highlighting for nanostructuring. FIG. 8 shows that formation of nanospheres on the zirconium dioxide surface was successful using the sputter deposition technology. The zirconium dioxide was sputter coated onto the sandblasted zirconium oxide, resulted in the nanostructure formation. The SEM images of sandblasted zirconium oxide surfaces before and after zirconium oxide sputter deposition. Bar=1 μm.

H. Increased Bone-Titanium Integration by Nanostructuring

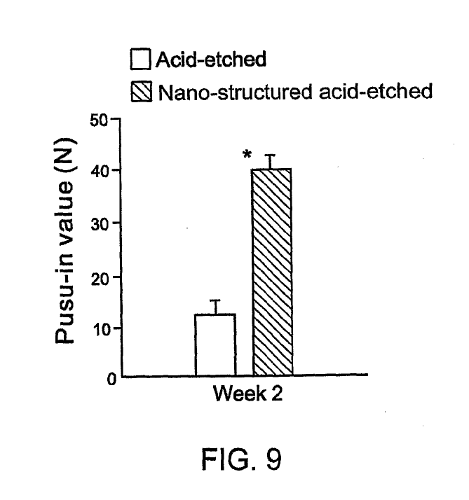

In vivo anchorage of titanium implants with or without nano-sphere structure was examined using the biomechanical implant push-in test. The acid-etched implants placed into the rat femur were pushed-in vertically, and the force at a point of breakage (maximum force on the load-displacement curves) was measured as a push-in value. The push-in value at 2 weeks post-implantation soared over 3 times after the nanostructuring (FIG. 9). In the test shown by FIG. 9, the acid-etched (HCl—H₂SO₄) titanium implants with or without the electron-beam physical vapor Ti deposition were placed into the rat femur, and the biomechanical stability of the implants were evaluated at 2 week post-implantation by measuring the breakage strength against push-in load. Data are shown as the mean±SD (n=5). The symbol “*” indicates that the data are statistically significant between the nanostructure implants and control implants, p<0.0001.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

1. An article comprising a substrate surface structure, the substrate surface structure comprising:
A nano structure formed on top of a microstructure on the surface of a substrate,
wherein the nano structure comprises a material which is not a ceramic,
wherein the nano structure comprises nano spheres or nanoparticles, and
wherein the nanospheres or nanoparticles do not form a continuous phase.

2. The article of claim 1, wherein the nanostructure comprises nanospheres or nanoparticles having a size in the range between about 1 nm to about 1,000 nm.

3. The article of claim 2, wherein the nanostructure comprises a metallic material selected from the group consisting of titanium, nickel, chromium, aluminum, zirconium, copper, zinc, ferrous, cadmium, lithium, titanium alloy, chromium-cobalt alloy, titanium dioxide, zirconium oxide, and combinations thereof.

4. The article of claim 1, wherein the nano structure comprises a non-metallic material.

5. The article of claim 4, wherein the non-metallic material is selected from the group consisting of a polymeric material, a semiconductor material, and combinations thereof.

6. The article of claim 1, wherein the substrate comprises a metallic material.

7. The article of claim 1, wherein the metallic material is selected from the group consisting of titanium, nickel, chromium, aluminum, zirconium, copper, zinc, ferrous, cadmium, lithium, titanium alloy, chromium-cobalt alloy, titanium dioxide, zirconium oxide, and combinations thereof.

8. The article of claim 1, wherein the substrate comprises a non-metallic material.

9. The article of claim 8, wherein the substrate comprises a non-metallic material selected from the group consisting of a polymeric material, a ceramic material, a semiconductor material, a bioglass, and combinations thereof.

10. The article of claim 1, wherein the nanostructure is generated by a process selected from the group consisting of electron-beam physical vapor deposition (EB-PVD), sputter coating, plasma spray, thermal vapor coating, laser vapor coating, photo vapor coating, chemical vapor deposition technology and combinations thereof.

11. The article of claim 1, wherein the microstructure is generated by a process selected from the group consisting of a physical process, a chemical process, or a combination thereof.

12. A process for forming a nanostructure on a substrate, comprising:
(a) forming a microstructure on the substrate, and
(b) forming a nanostructure on top of the microstructure,
wherein the nano structure comprises a material which is not a ceramic,
wherein the nanostructure comprises nano spheres or nanoparticles, and
wherein the nano spheres or nanoparticles do not form a continuous phase.

13. The process of claim 12, wherein the step (b) comprises
(1) forming a vapor of a nanostructuring material,
(2) depositing the vapor on a substrate having a microstructure surface, and
(3) forming a nanostructure of the nano structuring material on the substrate on the microstructure surface.

14. The process of claim 13, wherein the nanostructuring material is selected from the group consisting of a metallic material, a non-metallic material, and combinations thereof.

15. The process of claim 14, wherein the metallic material is selected from the group consisting of titanium, nickel, chromium, aluminum, zirconium, copper, zinc, ferrous, cadmium, lithium, titanium alloy, chromium-cobalt alloy, titanium dioxide, zirconium oxide, and combinations thereof, and
wherein the non-metallic material is selected from the group consisting of a polymeric material, a semiconductor material, and combinations thereof.

16. The process of claim 13, wherein the substrate comprises a material selected from the group consisting of a metallic material, a non-metallic material, and combinations thereof.

17. The process of claim 16, wherein the metallic material is selected from the group consisting of titanium, nickel, chromium, aluminum, zirconium, copper, zinc, ferrous, cadmium, lithium, titanium alloy, chromium-cobalt alloy, titanium dioxide, zirconium oxide, and combinations thereof and
wherein the non-metallic material is selected from the group consisting of a polymeric material, a bioglass, a ceramic material, a semiconductor material, and combinations thereof.

18. The process of claim 12, wherein the step (a) is by a process selected from the group consisting of a physical process, a chemical process, and combinations thereof.

19. The process of claim 18, wherein physical process is selected from the group consisting of machining, sand-blasting, and combinations thereof, and
wherein chemical process is selected from the group consisting of chemical etching, anodic oxidation, phot-etching, discharge processing and combinations thereof.

20. The article according to claim 1, which is a medical implant.

21. The article according to claim 1, which is a semiconductor article.

22. A method of treating, preventing, or ameliorating a medical condition in a mammal, comprising implanting in the mammal the article according to claim 20.
